(19) United States
(12) Patent Application Publication
(10) Pub. No.: US 2008/0214823 A1
(43) Pub. Date: Sep. 4, 2008

(54) Preparation of Montelukast

(75) Inventors: Pratap Reddy Padi, Hyderabad (IN); Satyanarayana Bollikonda, Hyderabad (IN); Alok Kumar Srivastava, Uttar Pradesh (IN); Ravi Kumar Kasturi, Hyderabad (IN); Rajender Reddy Jinna, Hyderabad (IN); Narsimha Naidu Mopidevi, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignee: Dr. Reddy's Laboratories Ltd., Bridgewater, NJ (US)

(21) Appl. No.: 11/996,453

(22) PCT Filed: Jul. 20, 2006
(86) PCT No.: PCT/US06/28431

§ 371 Date: Jan. 22, 2008

Related U.S. Application Data
(60) Provisional application No. 60/584,675, filed on Jul. 2, 2004.
(60) Provisional application No. 60/566,603, filed on Apr. 30, 2004.

(30) Foreign Application Priority Data
Jul. 20, 2005 (IN) 966/CHE/2005
Mar. 14, 2006 (IN) 455/CHE/2006

(51) Int. Cl. C07D 215/18

(52) U.S. Cl. 546/165

(57) ABSTRACT

A process for preparing amorphous montelukast sodium comprises removing solvent from a solution comprising montelukast sodium using agitated thin film drying.

### INTRODUCTION TO THE INVENTION

The present invention relates to substantially pure montelukast, its pharmaceutically acceptable salts and a process for its preparation. The process of the present invention is suitable for industrial scale production.

Montelukast is described chemically as [R-(E)]-1-[[[1-[3-[2-(7-chloro-2-quinolinyl)ethenyl]phenyl]-3-[2-(1-hydroxy-1-methylethyl)phenyl]propyl]thio]methyl]cyclopropaneacetic acid (hereinafter referred to by its adopted name “montelukast”) and is structurally represented by Formula I.

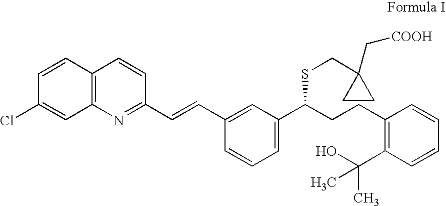

Montelukast is a selective and orally active leukotriene receptor antagonist that inhibits the cysteinyl leukotriene CysLT₁ receptor and is useful in the treatment of asthma as well as other conditions mediated by leukotrienes, such as inflammation and allergies.

Montelukast is commercially available in the market in products sold under the trademark SINGULAIR as chewable tablets. Each 10 mg, 4 mg, or 5 mg chewable SINGULAIR tablet respectively contains 10.4 mg, 4.2 mg, and 5.2 mg of montelukast sodium, which is equivalent to 10, 4, and 5 mg of montelukast respectively.

U.S. Pat. No. 5,565,473 discloses generically and specifically montelukast and its related compounds along with their pharmaceutically acceptable salts.

Processes for preparation of montelukast have also been described in U.S. Pat. Nos. 5,614,632 and 5,523,477, U.S. Patent Application Publication Nos. 2005/0234241 A1, 2005/0256156 A1, and 2005/0107612, and International Application Publication Nos. WO 2005/105749, WO 2005/000807, and WO 2004/108679.

The synthesis of montelukast involves many synthetic steps in which undesired products are obtained. Therefore, the final product can be contaminated not only with the undesired products derived from the last synthetic step of the process but also with compounds that were formed in previous steps. These products should be removed from the final product in order to meet the ICH specifications for purity.

Regulatory authorities worldwide require that drug manufacturers isolate, identify and characterize the impurities in their products. Moreover, it is required to control the levels of these impurities in the final drug compound obtained by the manufacturing process and to ensure that the impurity is present in the lowest possible levels.

Hence, there is a need for a purification method for montelukast that uses a simple and commercially viable process while achieving the desired purity. Even though crystallization is known to be the simplest process that can be used for purification of organic compounds, many of the impurities are hard to remove as they co-crystallize with montelukast salts. The right choice of solvents for crystallization plays a major role in removing the desired impurities from the compound and therefore purifying it. The solvent of choice should effectively remove the impurity without sacrificing the yield.

The present invention provides a process for the preparation of substantially pure montelukast sodium free of any process related impurities and also free of residual organic solvents. The process of the present invention can be practiced on an industrial scale, and also can be carried out without sacrifice of overall yield based on the starting materials employed.

### SUMMARY OF THE INVENTION

The present invention relates to substantially pure montelukast and its pharmaceutically acceptable salts, and a process for their preparation.

In one aspect, the present invention provides substantially pure montelukast or its pharmaceutically acceptable salts.

In another aspect, the present invention provides a process for the purification of montelukast acid to substantially remove the montelukast styrene and montelukast deschloro impurities.

In an embodiment, a process for preparing montelukast acid substantially free of the montelukast styrene and montelukast deschloro impurities comprises the steps of:

a) providing a mixture of montelukast acid with a suitable solvent;

b) optionally treating the mixture with activated charcoal;

c) isolating the solid from the mixture;

d) recovering the separated solid.

In yet another aspect, the invention provides a process for purification of montelukast amine salts to remove the montelukast sulfoxide impurity.

In an embodiment, a process for preparing montelukast amine salt substantially free of montelukast sulfoxide impurity comprises the steps of:

a) providing a solution of montelukast amine salt;

b) optionally treating the solution with activated charcoal;

c) crystallizing the solid from the solution;

d) recovering the separated solid.

Still another aspect of the invention provides a process for the preparation of montelukast sodium substantially free of residual organic solvents.

In an embodiment, a process for preparing montelukast sodium substantially free of residual organic solvents comprises the steps of:

a) providing a solution of montelukast sodium;

b) removing the solvent from the solution obtained in step a);

c) drying the solid using a suitable technique;

A further aspect of the invention provides a method of packaging of montelukast sodium that provides improved stability to montelukast sodium upon storage.

In a still further aspect, the present invention provides a pharmaceutical composition comprising substantially pure montelukast or its pharmaceutically acceptable salts along with one or more pharmaceutically acceptable carriers, excipients or diluents.

An aspect of the invention includes a process for preparing amorphous montelukast sodium comprising removing solvent from a solution comprising montelukast sodium using agitated thin film drying.

An aspect of the invention includes a process for preparing montelukast sodium, comprising:

dissolving montelukast in a solvent and recrystallizing montelukast;

reacting recrystallized montelukast with t-butyl amine to form a salt and recovering solid product;

dissolving a t-butyl amine salt of montelukast in a solvent and recrystallizing a t-butyl amine salt of montelukast; and

reacting a recrystallized t-butyl amine salt of montelukast with sodium hydroxide.

An aspect of the invention includes a method for packaging montelukast sodium, comprising:

placing montelukast sodium in a sealed container under an inert atmosphere;

placing the sealed container, a desiccant, and an oxygen adsorbent in a second sealed container;

placing the second sealed container in a triple laminated bag and sealing; and

enclosing the triple laminated bag in a closed high density polyethylene (“HDPE”) container.

### DETAILED DESCRIPTION OF THE INVENTION

The present invention relates to substantially pure montelukast or its pharmaceutically acceptable salts and a process for its preparation.

In one aspect, the invention provides substantially pure montelukast or its pharmaceutically acceptable salts.

By “substantially pure montelukast” it is meant that montelukast acid or any of the pharmaceutically acceptable salts of montelukast prepared in accordance with the present invention contains less than about 0.5%, or less than about 0.1% of the corresponding impurities like montelukast styrene, montelukast deschloro and montelukast sulfoxide impurities as characterized by a high performance liquid chromatography (“HPLC”) chromatogram obtained from a mixture comprising the desired compound and one or more of the said impurities. The percentage here refers to the area-% of the peaks representing the said impurities.

The pharmaceutically acceptable salts of montelukast refer to salts prepared form pharmaceutically acceptable non-toxic bases including inorganic bases and organic bases, or acids including inorganic and organic acids.

Salts derived from inorganic bases include aluminium, ammonium, calcium, copper, ferric, ferrous, lithium, magnesium, manganic salts, manganous, potassium, sodium, zinc, and the like. Salts derived from organic non-toxic bases include, salts of primary, secondary, and tertiary amines, substituted amines including naturally occurring substituted amines.

As used herein “montelukast styrene impurity” refers to [R-(E)]-1-[[[1-[3-[2-(7-chloro-2-quinolinyl)ethenyl]phenyl]-3-[2-[1-(1-methyl)ethenyl)]phenyl]propyl]thio]methyl]cyclopropaneacetic acid represented by Formula II;

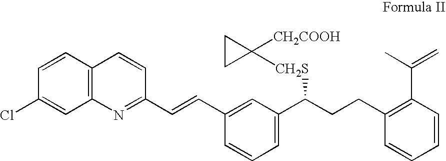

“montelukast des-chloro impurity” refers to [R-(E)]-1-[[[1-[3-[2-(2-quinolinyl)ethenyl]phenyl]-3-[2-(1-hydroxy-1-methylethyl)phenyl]propyl]thio]methyl]cyclopropane acetic acid represented by Formula III; and

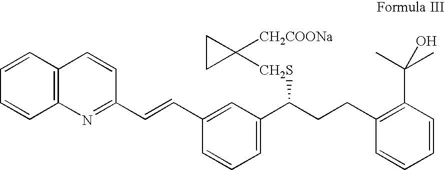

“montelukast sulfoxide impurity” refers to [R-(E)]-1-[[[1-[3-[2-(7-chloro-2-quinolinyl)ethenyl]phenyl]-3-[2-(1-hydroxy-1-methylethyl)phenyl]propyl]sulfoxo]methyl]cyclopropane acetic acid represented by Formula IV.

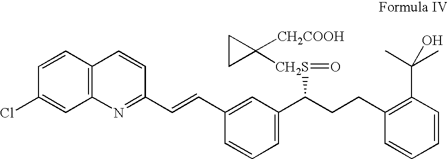

Montelukast having a reduced level of impurities typically also contains residual solvents. For purposes of the present invention, any residual solvents in purified montelukast are also considered as impurities. Residual solvents can be quantified by application of known chromatographic techniques.

Another aspect of the invention provides a process for the purification of montelukast acid to remove the montelukast styrene and montelukast deschloro impurities.

In an embodiment, the process for the purification of montelukast acid to remove montelukast styrene and montelukast deschloro impurities comprises the steps of:

a) providing a mixture of montelukast acid with a suitable solvent;

b) optionally, treating the mixture with activated charcoal;

c) crystallizing the solid from the mixture;

d) recovering the separated solid.

Step a) involves providing a mixture of montelukast acid in a suitable solvent.

Montelukast acid obtained using any of the processes described in the art, or the acid obtained by following a process similar to the one described in U.S. Patent Application Publication No. US 2005/0234241 A1 can be purified using the process of the present invention.

The mixture of montelukast may be obtained by suspending montelukast acid in a suitable solvent, or such a mixture may be obtained directly from a reaction in which montelukast acid is formed.

When the mixture is prepared by suspending montelukast acid in a suitable solvent, any form of montelukast acid such as any crystalline or amorphous form including any salts, solvates and hydrates may be utilized for preparing the solution.

Suitable solvents which can be used for suspending montelukast acid, include but are not limited to: alcohols such as methanol, ethanol, isopropyl alcohol, n-propanol, and the like; ketones such as acetone, ethyl methyl ketone, methyl isobutyl ketone and the like; hydrocarbons such as toluene, xylene, n-heptane, cyclohexane, and the like; or mixtures thereof or their combinations with water in various proportions.

The temperatures for preparation of the mixture can range from about 20 to 120° C. depending on the solvent used. Any other temperature is also acceptable as long as the stability of montelukast is not compromised.

The quantity of solvent used for preparing the mixture depends on the nature of solvent and the temperature adopted for preparing the mixture. The concentration of montelukast acid in the mixture may generally range from about 0.1 to about 10 g/ml in the solvent.

The mixture can be in the form of a clear solution or a suspension.

Step b) involves the treatment of the mixture obtained in step a) with activated charcoal.

The mixture obtained in step a) can be optionally treated with activated charcoal to enhance the color of the compound followed by filtration through a medium such as through a flux calcined diatomaceous earth (Hyflow) bed to remove the carbon.

The carbon treatment can be given either at the temperatures of the preparation of the mixture or after cooling the solution to lower temperatures.

Step c) involves isolation of the solid from the mixture.

For isolation to occur, the reaction mass may be maintained further at temperatures lower than the concentration temperatures such as for example below about 10° C. to about 25° C., for a period of time as required for a more complete isolation of the product. The exact cooling temperature and time required for complete isolation can be readily determined by a person skilled in the art and will also depend on parameters such as concentration and temperature of the solution or slurry.

Optionally isolation may be enhanced by methods such as cooling, partial removal of the solvent from the mixture, by adding an anti-solvent to the reaction mixture or a combination thereof.

Step d) involves recovering the separated solid.

The method by which the solid material is recovered from the final mixture, with or without cooling below the operating temperature, can be any of techniques such as filtration by gravity, or by suction, centrifugation, and the like. The crystals so isolated will carry a small proportion of occluded mother liquor containing a higher percentage of impurities. If desired the crystals can be washed on the filter with a solvent to wash out the mother liquor.

In a particular embodiment of the invention the above described process of the invention can be adapted to form the basis of a continuous crystallization process. The purity of the product obtained in step d) is checked to see the percentage of the impurities. If the impurities are not reduced to the required level of below 0.1 area-% by HPLC, then, the steps a) to d) are repeated with the wet material obtained in step d). When the desired purity is attained at step d), the cycle is stopped.

Thus there is established a cycle of operations, which can be, repeated indefinitely thereby adapting the process of the invention to a continuous process with obvious attendant advantages on the commercial scale.

The wet cake obtained in step d) may optionally be further dried. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any desired time periods from about 1 to 20 hours.

The purified montelukast acid obtained above contains less than 0.1 area-%, or less than 0.05 area-%, of either of the montelukast styrene and montelukast deschloro impurities. The purified montelukast acid may be converted to its amine salt by processes known in the art or by a process comprising:

a) providing a mixture of montelukast acid with a suitable solvent;

b) adding the amine to the mixture obtained in step a);

c) isolating the amine from the mixture;

Step a) involves providing a mixture of montelukast acid in a suitable solvent.

The mixture of montelukast acid may be obtained by suspending montelukast acid in a suitable solvent, or such a mixture may be obtained directly from a reaction in which montelukast acid is formed.

When the mixture is prepared by dissolving montelukast acid in a suitable solvent, any form of montelukast acid such as any crystalline or amorphous form including any salts, solvates and hydrates may be utilized for preparing the solution.

Suitable solvents which can be used for the preparation of the mixture of montelukast acid include, but are not limited to; alcoholic solvents like methanol, ethanol, isopropyl alcohol and the like, ketonic solvents such as acetone, ethylmethyl ketone, methyl isobutyl ketone and the like hydrocarbon solvents such as toluene, xylene and the like; nitrile solvents such as acetonitrile, propionitrile and the like; or mixtures thereof in various proportions.

Step b) involves adding the amine to the mixture obtained in step a);

The organic non-toxic amines which can be used for the preparation of montelukast amine salts include primary, secondary, and tertiary amines, substituted amines including naturally occurring substituted amines, cyclic amines, and basic ion exchange resins, such as arginine, batanine, caffeine, choline, N,N′-dibenzylenediamine, diethylamine, triethylamine, trimethylamine, tripropylamine, and the like.

The amine can be added to the reaction mass at temperatures lower than the dissolution temperatures or at the dissolution temperatures. The temperatures for addition of the amine can range from about 0° C. to about 60° C. or more.

After addition of the amine the reaction mass may be maintained further at temperatures lower than the dissolution temperatures such as for example below about 10° C. to about 25° C., for a period of time as required for a more complete isolation of the product. The exact cooling temperature and time required for complete precipitation can be readily determined by a person skilled in the art.

Optionally, small amounts of seeding crystals montelukast amine salt may be added to the reaction mixture. Preferably, small amounts are about 1 to 20 weight %, more preferably about 5 weight %. Seeding crystals may be added before or, where appropriate, after the step initiating the precipitation.

Step c) involves isolating the amine from the solution;

The amine salt can be isolated from the reaction mass using techniques such as filtration by gravity, or by suction, centrifugation, and the like. The crystals so isolated will carry a small proportion of occluded mother liquor. If desired the crystals can be washed on the filter with a solvent.

Optionally, the wet solid obtained can be dried. Drying can be carried out at reduced pressures, such as below 200 mm Hg or below 50 mm Hg, at temperatures of about 50° C. to about 80° C. The drying can be carried out for any desired or required time periods, times about 1 to 20 hours being suitable for preparing some products.

Yet another aspect of the present invention provides a process for purification of montelukast amine salts to remove the montelukast sulfoxide impurity.

In an embodiment, the process for preparing montelukast amine salt free of montelukast sulfoxide impurity comprises the steps of:

a) providing a solution of montelukast amine salt in a suitable solvent;

b) optionally treating the solution with activated charcoal;

c) crystallizing the solid from the solution;

d) recovering the separated solid.

Step a) involves providing a solution of montelukast amine salt in a suitable solvent.

Montelukast amine salt for the purpose of purification may be one prepared according to the processes described in the prior art, or using a process similar to the one described above.

The solution of montelukast amine salt may be obtained by dissolving the montelukast amine in a suitable solvent, or such a solution may be obtained directly from a reaction in which montelukast amine is formed.

When the solution is prepared by dissolving montelukast amine in a suitable solvent, any form of montelukast amine salt such as any crystalline or amorphous form including any salts, solvates and hydrates may be utilized for preparing the solution.

Suitable solvents which can be used for dissolving montelukast amine include but are not limited to: ketones such as acetone, ethyl methyl ketone, methyl isobutyl ketone and the like; hydrocarbons such as toluene, xylene, n-heptane, cyclohexane, n-hexane and the like; nitrites such as acetonitrile, propionitrile and the like; or mixtures thereof or their combinations with water in various proportions.

The dissolution temperatures can range from about 20 to 120° C. depending on the solvent used for dissolution. Any other temperature is also acceptable as long as the stability of montelukast is not compromised and a clear solution is obtained.

The quantity of solvent used for dissolution depends on the solvent and the dissolution temperature adopted. The concentration of montelukast amine in the solution may generally range from about 0.1 to about 10 g/ml in the solvent.

Step b) involves the treatment of the solution obtained in step a) with activated charcoal.

The solution obtained in step a) can be optionally treated with activated charcoal to enhance the color of the compound followed by filtration through a medium such as through a flux calcined diatomaceous earth (Hyflow) bed to remove the carbon.

The carbon treatment can be given either at the dissolution temperatures or after cooling the solution to lower temperatures.

Step c) involves crystallization of the solid from the solution.

For crystallization to occur, the reaction mass may be maintained further at temperatures lower than the concentration temperatures such as for example below about 10° C. to about 25° C., for a period of time as required for a more complete isolation of the product. The exact cooling temperature and time required for complete crystallization can be readily determined by a person skilled in the art and will also depend on parameters such as concentration and temperature of the solution or slurry.

Optionally crystallization may be initiated by methods such as cooling, seeding, partial removal of the solvent from the solution, by adding an anti-solvent to the solution or a combination thereof.

Step d) involves recovering the separated solid.

The method by which the solid material is recovered from the final mixture, with or without cooling below the operating temperature, can be any of techniques such as filtration by gravity, or by suction, centrifugation, and the like. The crystals so isolated will carry a small proportion of occluded mother liquor. If desired the crystals can be washed on the filter with a solvent.

In a particular embodiment of the invention the above described process of the invention can be adapted to form the basis of a continuous crystallization process. The purity of the product obtained in step d) is checked to see the percentage of montelukast sulfoxide impurity. If the impurity is not reduced to the required levels of below 0.1 area-% by HPLC, then, the steps a) to d) are repeated with the wet material obtained in step d). When the desired purity is attained at step d), the cycle is stopped.

Thus there is established a cycle of operations which can be repeated indefinitely thereby adapting the process of the invention to a continuous process with obvious attendant advantages on the commercial scale.

The wet cake obtained in step d) may optionally be further dried. Drying can be suitably carried out in a tray dryer, vacuum oven, air oven, or using a fluidized bed drier, spin flash dryer, flash dryer and the like. The drying can be carried out at temperatures of about 35° C. to about 70° C. The drying can be carried out for any desired time periods, times about 1 to 20 hours.

The purified montelukast amine salt obtained above contains less than 0.1 area-% or less than 0.05 area-% of the montelukast sulfoxide impurity.

The purified montelukast amine salt obtained by the process of the invention can be used without further treatment for the preparation of montelukast sodium.

Still another aspect of the invention provides a process for the preparation of montelukast sodium to get montelukast sodium free of residual organic solvents.

The process for preparing montelukast sodium free of residual organic solvents comprises the steps of:

a) providing a solution of montelukast sodium in a suitable solvent;

b) removing the solvent from the solution obtained in step a);

c) drying the solid using a suitable technique;

Step a) involves providing a solution of montelukast sodium.

The solution of montelukast sodium can be obtained starting from its amine salt using a process given in the prior art or a process similar to the one given in U.S. Patent Application Publication No. 2005/0234241 A1 (Example 3).

Suitable solvents which can be used for the preparation of the montelukast sodium solution include, but are not limited to; alcohols such as methanol, ethanol, isopropyl alcohol and the like, ketones such as acetone, ethyl methyl ketone, methyl isobutyl ketone and the like; hydrocarbons such as toluene, xylene and the like; nitrites such as acetonitrile, propionitrile and the like; or mixtures thereof or their combination with water in various proportions without limitation.

The process for obtaining a solution of montelukast sodium typically involves breaking of the montelukast amine salt using an acid followed by treatment with a sodium salt in a suitable solvent to form the montelukast sodium solution.

Step b) involves removing the solvent from the solution obtained in step a).

Removal of the solvent may be carried out suitably using techniques such as evaporation, atmospheric distillation, distillation under vacuum, and the like.

Distillation of the solvent may be conducted under vacuum, such as below about 100 mm Hg to below about 600 mm Hg, at elevated temperatures such as about 20° C. to about 70° C. Any temperature and vacuum conditions can be used as long as there is no increase in the impurity levels of the product.

Suitable techniques which can be used for the solvent removal include, distillation using a rotational evaporator device such as a Buchi Rotavapor, spray drying, agitated thin film drying (“ATFD”), and the like.

An embodiment of the invention involves the removal of the solvent using an agitated thin film drying-vertical (“ATFD-V”) technique.

The ATFD-V technique uses high vacuum along with elevated temperatures which allows operation at lower temperatures. This allows for a short residence time for the product in the drier. The required evaporation can be achieved in a single pass, avoiding product recirculation and possible degradation. The operating pressures are from atmospheric down to 1 mbar. The equipment can be operated at a wide range of temperatures, such as 25 to 350° C. or more.

The concentration, solvent type, temperature, vacuum, and feeding rate are set to combinations where the montelukast sodium salt coming from the inlet precipitates essentially instantly.

The process frequently is carried out at temperatures that are below the atmospheric pressure boiling point of the solvent, such as about 35° C. to about 60° C., under a reduced pressure such as about 400 to about 740 mm Hg. These dryers are indirectly heated and therefore air does not come in contact with the product, thus avoiding the formation of the sulfoxide impurity. The temperature and pressure conditions can vary depending on properties of the solvent that is being removed, and can be higher or lower than the ranges mentioned.

The solution of montelukast sodium may be added dropwise or continuously to the drying chamber. The rate of flow may range from 10 to 50 cm³/hour/inlet. These and other parameters are well known to a person skilled in the art of drying using ATFD, and will vary depending upon characteristics of the actual apparatus being used.

ATFD-V helps in evaporating solvents by using heat transfer across the walls and prevents the growth of crystals and particles that can trap the solvent at higher levels. The resulting montelukast sodium salt is a solid amorphous form having a solvent content lower than for the compound obtained from other techniques of evaporation like the Buchi Rotavapor or spray drier.

The yields obtained using this technique are superior to those obtained using other techniques.

Step c) involves drying of the isolated product of step b) to afford montelukast or its pharmaceutically acceptable salts in the amorphous form.

Drying can be carried out under reduced pressure until the residual solvent content reduces to within the limits given by the ICH guidelines. The solvent level depends on the type of solvent but is not more than about 5000 ppm, or about 4000 ppm, or about 3000 ppm.

The drying can be carried out at reduced pressures, such as below 200 mm Hg or below 50 mm Hg, at temperatures of about 40° C. to about 80° C. The drying can be carried out for any desired time periods, times about 1 to 20 hours being suitable for preparing some products.

Drying can be suitably carried out in a tray dryer, vacuum oven, air oven, or using a fluidized bed drier, spin flash dryer, flash dryer and the like.

This drying technique lowers the solvent content to the required limits set by ICH guidelines. The drying process is easily scalable for industrial purposes and the results obtained are reproducible.

The dried product can optionally be milled to get a desired particle size. Milling or micronization can be performed prior to drying, or after the completion of drying of the product. The milling operation reduces the size of particles and increases surface area of particles by colliding particles with each other at high speeds.

Drying is more efficient when the particle size of the material is smaller and the surface area is higher, hence milling can be performed prior to the drying operation.

Milling can be done suitably using jet milling equipment like an air jet miller, or using other conventional milling equipment.

Montelukast prepared according to the process of the present invention is also free of impurities at 0.156, 0.77, 0.84, 1.20, and 2.16 RRT's as measured by HPLC.

Still another aspect of the invention provides a method of packaging of montelukast sodium that provides improved stability to montelukast sodium upon storage.

It has been observed that montelukast sodium is an unstable substance, which is susceptible to moisture and picks up moisture easily when exposed to atmosphere. Also the reaction of montelukast with atmospheric oxygen leads to the formation of the sulfoxide impurity.

The susceptibility of montelukast to moisture leads to deviation of the drug product from regulatory purity requirements even prior to the product reaching the patient.

Therefore, to provide consistent purity of montelukast the packaging conditions have been modified such that they delay or prevent the pick up of moisture, and formation of sulfoxide impurity by the product.

A packaging and storage process for stabilizing hygroscopic active substance montelukast sodium comprises.

a) placing montelukast sodium in a sealed container under an inert atmosphere;

b) placing the sealed container, a desiccant, and an oxygen adsorbent, in a second sealed container;

c) placing the second sealed container in a triple laminated bag followed by sealing;

d) enclosing the triple laminated bag in a closed high-density polyethylene (“HDPE”) container.

Step a) involves storing the active substance in an inert atmosphere.

The inert atmosphere can be provided using any of the inert gases such as nitrogen, argon, and the like. The gas should not react with montelukast sodium and should be free from moisture.

The inert atmosphere can be provided to the compound which is kept in a polythene bag, or has been stored in a more rigid container. The bag or container which is used to provide the inert atmosphere to montelukast is sealed air tight after providing the inert atmosphere.

If the container which is used to provide the inert atmosphere to montelukast is transparent and exposes the product to light, then it can be covered using a non-transparent material.

Step b) involves placing the bag or container containing montelukast sodium, a moisture adsorbent (desiccant), and an oxygen adsorbent into a second bag or more rigid container.

The moisture adsorbent and the oxygen adsorbent are included in order to absorb any moisture and oxygen which enters the packaging.

Suitable moisture adsorbents which can be used in the present invention include, but are not limited to molecular sieve zeolites, high silica zeolites, having a high silica/alumina ratio of 25 or more, such as ZSM-5 (made by Mobil Oil Co., silica/alumina ratio of 400), silicalite, USY (Ultra Stable Y type zeolite, by PQ Corp., silica/alumina ratio of 78), mordenite and the like, a low silica system zeolite such as Ca—X type zeolite, Na—X type zeolite, silica super fine granulated particle (for example, particle having an average particle size of 1.5 mm which has been obtained by granulating the silica super fine particle having a size of 0.1 μm or less), silica gel, γ-alumina, and the like.

Suitable oxygen adsorbents which can used include, but are not limited to CuO (that has been activated by reduction with hydrogen) on an inorganic oxide, sachet of Ageless Z 200 which reduces the oxygen concentration in a sealed container to below 0.01% creating a very low-oxygen environment. Ageless sachets contain fine iron powder covered with sea salt and a natural zeolite impregnated with a NaCl solution. One sachet of Ageless Z 2000 absorbs 2000 ml of oxygen (the oxygen from 10 L of air) and other similar oxygen absorbents can be used.

Step c) involves placing a second bag or container in a triple laminated bag followed by sealing.

The packing containing the compound and the oxygen and moisture adsorbents are kept in a triple laminated bag, having layers of polyethylene terephthalate film, aluminum foil, and linear low-density polyethylene film. The triple laminated bag provides protection to the contents from oxygen, water vapor, light, and other contaminants.

Optionally an additional moisture adsorbent is put into the triple laminated bag as an additional precaution to adsorb any moisture which enters it.

The triple laminated bag is heat sealed to prevent the entry of any contaminants. The heat sealing can be done using a vacuum nitrogen sealer (VNS) for effective sealing.

Step d) involves storing the triple laminated bag in a HDPE container.

It has been found that the above packaging and storage process provides substantially pure montelukast sodium, which is stable during storage and does not undergo agglomeration, and also results in minimizing sulfoxide impurity.

In a still further aspect, the present invention provides a pharmaceutical composition comprising substantially pure montelukast or its pharmaceutically acceptable salts along with one or more pharmaceutically acceptable carriers, excipients or diluents.

The pharmaceutical composition comprising substantially pure montelukast or its pharmaceutically acceptable salts along with one or more pharmaceutically acceptable carriers of this invention may further formulated as: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared by direct blending, dry granulation or wet granulation or by extrusion and spheronization. Compositions may be presented as uncoated, film coated, sugar coated, powder coated, enteric coated or modified release coated. Compositions of the present invention may further comprise one or more pharmaceutically acceptable excipients.

Pharmaceutically acceptable excipients that find use in the present invention include, but are not limited to: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl methylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethyl cellulose, methyl cellulose, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants and the like.

In the compositions of present invention montelukast or its pharmaceutically acceptable salts is a useful active ingredient in the range of 0.5 mg to 50 mg, or 1 mg to 25 mg.

Certain specific aspects and embodiments of this invention are described in further detail by the examples below, which examples are not intended to limit the scope of the appended claims in any manner.

### EXAMPLE 1

### Determination of Impurities in Montelukast Sodium

Determining the level of impurities in montelukast and its salts using HPLC. The HPLC analysis conditions are as described in Table 1.

TABLE 1
HPLC method for detecting the level of the impurities.

| Column and | Hypersil BDS-C18, 100 × 4.6 mm ID, 3μ. | | |
|---|---|---|---|
| Packing: | | | |
| Buffer: | 3.9 g NaH₂PO₄•H₂O was taken in 1000 mL of MQ water | | |
| | and adjust pH to 3.7 with orthophosphoric acid. | | |
| Mobile | Mixed buffer and acetonitrile in the ratio 800:200 v/v. | | |
| Phase A: | | | |
| Mobile | Mixed buffer and acetonitrile in the ratio 200:800 v/v. | | |
| Phase B: | | | |
| Gradient: | Time (in minutes) | Event | Value |
| | 0.01 | B. Conc. | 47 |
| | 35 | B. Conc. | 95 |
| | 58 | B. Conc. | 95 |
| | 62 | B. Conc. | 47 |
| | 70 | B. Conc. | 47 |

| | |
|---|---|
| Temperature: | 27° C. |
| Injection | 20 μL |
| volume: | |
| Flow rate: | 1.0 ml per minute |
| Detector: | 225 nm |
| Diluent: | Acetonitrile: water (60:40) |
| Sample | 0.5 mg/mL in diluent |
| concentration: | |
| Run time: | 70 minutes |

Note:
To calculate the % area of sulfoxide, area of main peak of sulfoxide and the area of the corresponding diastereomer of it at RRT 0.46 should sum up and calculate as total sulfoxide area percentage.

| IMPURITY NAME | RRT |
|---|---|
| Montelukast styrene impurity | 1.57 |
| Montelukast des-chloro impurity | 0.67 |
| Montelukast sulfoxide impurity | 0.47 |

### EXAMPLE 2

### Determination of Residual Solvents in Montelukast Sodium

TABLE 2
Gas Chromatography method for detecting
residual solvent content:

| | |
|---|---|
| Column and | DB-WAX capillary column 30 m length, 0.53 mm ID, |
| Packing: | 1.0 μm film thickness or equivalent. |
| Column Flow: | 20 cm/second. |
| Injector | 100° C. |
| Temperature: | |
| Detector (FID) | 230° C. |
| Temperature: | |
| Injection | Split |
| mode: | |

Method of analysis:
Split ratio: 1:5.
Injection volume: 1.0 ul.

### Diluent: Dimethylsulfoxide.

Make up gas: 30 ml per minute.
Oven temperature program:
Oven temperature is held at 40° C. for 10 minutes, then raised to 110° C. at the rate of 6° C. per minute, held at 110° C. for 12 minutes then raised to 220° C. at the rate of 35° C. per minute, held at 220° C. for 15 minutes.
Sample Preparation: 200 mg/10 ml dimethylsulfoxide.

### EXAMPLE 3

### Preparation of Montelukast Acid (Formula I)

100 g of 2-(2-(3(S)-(3-(2-(7-chloro-2-quinolinyl)ethenyl) phenyl)-3-hydroxypropyl)phenyl)-2-propanol and 500 ml of toluene were charged into a round bottom flask equipped with Dean-Stark apparatus. The resultant suspension was heated to 112° C. followed by stirring for 1 hour for removal of unwanted water along with the solvent from the reaction solution. Resultant residue was cooled to about 60° C. and 920 ml of acetonitrile was charged to the residue followed by further cooling to −15° C. 42.01 ml of diisopropylethylamine was added to the residue and was stirred for about 45 minutes. 16.91 ml of methanesulfonyl chloride was added drop wise to the reaction mass in 30 minutes followed by stirring for about 9 hours. Separated solid was filtered and the solid was washed with 200 ml of acetonitrile cooled to a temperature of 5° C. followed by washing with 200 ml of cyclohexane cooled to a temperature of 5° C. The solid obtained was dried at −15° C. under vacuum for 1 hour.

33.3 g of (1-mercaptomethyl)cyclopropaneacetonitrile and 500 ml of N,N-dimethylformamide were charged in another round bottom flask followed by cooling to about −15° C. 218.5 ml of n-butyl lithium in n-hexane was added drop wise to the above reaction mass in about 30 minutes under N₂ atmosphere. The reaction mass was maintained at −15° C. for 45 minutes, followed by charging of the mesylated compound under N₂ atmosphere. Resultant reaction mixture was stirred for 60 minutes. Reaction mass was quenched using 1000 ml of saturated sodium chloride solution (320 g sodium chloride in 1000 ml water) in 30 minutes followed by allowing the temperature of the reaction to raise to 29° C. The reaction mass was extracted with 1800 ml of toluene followed by separation of the organic layer. The total organic layer was washed with 4×1200 ml of water.

The organic layer was separated and distilled completely at about 55° C. under a vacuum of 300 mm Hg to give 105.2 g of crude compound. The obtained crude and 50 ml of toluene were charged in a clean and dry round bottom flask equipped with a Dean-Stark apparatus, and was heated to 111° C. (azeotropic reflux) to remove toluene azotropically, followed by stirring the reaction mass for about 12 to 15 hrs at about 130° C. Reaction completion was checked using thin layer chromatography. After the reaction was completed, the reaction mass was cooled to about 90° C. and the caustic lye layer was decanted. 2500 ml of preheated water (heated to 90° C.) was charged and was stirred for 1 hour for homogenous solution. pH of resultant reaction solution was adjusted to 11 by the addition of 30 ml of acetic acid under stirring. Reaction mass was washed with 4×600 ml of toluene and again pH was adjusted to 5.2 by the addition of 11.2 ml of acetic acid. Resultant reaction mass was cooled to about 28° C. and the organic and aqueous phases were separated. Aqueous layer was extracted with 2×400 ml of toluene, organic and aqueous layers were separated. The combined organic layer was washed with 5×500 ml of water. The organic layer was distilled completely at about 55° C. under a vacuum of 300 mm Hg. 100 ml of toluene was charged to the resultant residue and was stirred for 2 hours at about 28° C. The resultant homogenous solution was cooled to 2° C. for about 2 hours. Separated solid was filtered and the solid obtained was washed with 10 ml toluene cooled to a temperature of 5° C. Solid was dried at about 70° C. for 5 hours to afford 44.6 g of title compound.

### EXAMPLE 4

### Purification of Montelukast Acid

58.8 liters of methanol and 16.8 kg of crude montelukast acid (purity: 95.23%) were taken into a reactor and the reaction mass was heated to 62° C. The reaction mass was maintained at 62° C. for 30 minutes. Then the reaction mass was cooled to 30° C. and maintained for 6 hours. The reaction mass was further cooled 4° C. and maintained for 6 hours. The reaction mass was centrifuged and the centrifuged cake was washed with 16.8 liters of methanol chilled to a temperature of 2° C. The wet cake was taken into another reactor and 42 liters of methanol was added to it. The reaction mass was heated to 62° C. The reaction mass was maintained at 62° C. for 30 minutes. Then the reaction mass was cooled to 27° C. and maintained for 6 hours. The reaction mass was further cooled to 2° C. and maintained for 6 hours. The reaction mass was centrifuged and the centrifuged cake was washed with 16.8 liters of methanol chilled to a temperature of 2° C. The wet compound was dried at 64° C. for 10 hours to obtain 12.2 kg (72%) of the title compound.

Purity by HPLC: 98.7%

% of montelukast styrene impurity: 0.1%

% of montelukast deschloro impurity: 0.05.

### EXAMPLE 5

### Preparation of montelukast tertiary butyl amine salt

34 g of montelukast acid and 340 ml of acetone were charged in a clean and dry round bottom flask and was stirred for 15 min. 7.99 ml of tertiary butylamine was added to the above homogenous reaction solution followed by seeding with 0.34 g of montelukast tertiary butyl amine salt. Resultant suspension was stirred for about 45 minutes and 170 ml of acetone was added under stirring. Resultant reaction suspension was stirred for about 4 hours. Separated solid was filtered and the solid obtained was washed with 17 ml of acetone. Solid obtained was dried at about 60° C. for 3 hours to yield 36.5 g of title compound.

### EXAMPLE 6

### Purification of Montelukast Tertiary Butyl Amine Salt

71 liters of toluene and 12.9 kg of montelukast tertiary butyl amine salt were taken into a reactor and the mass was heated to 82° C. Carbon treatment was given to the mass at 82° C. The mass was filtered through a candy filter in the hot condition. The carbon bed was washed with 45.15 liters of pre-filtered toluene heated to a temperature of 82° C. The combined filtrate was taken into another reactor and maintained at 28° C. for 10 hours. The mass was then filtered through a Nutsche filter and the solid was washed with 6.45 liters of toluene. The wet material was taken into another reactor and 58 liters of toluene was added to it. The reaction mass was heated to 82° C. and checked for clear dissolution. After clear dissolution was obtained, carbon treatment was given to the mass at 82° C. The mass was filtered through a candy filter in the hot condition. The carbon bed was washed with 45.15 liters of pre-filtered toluene heated to a temperature of 82° C. The combined filtrate was taken into another reactor and maintained at 28° C. for 10 hours. The mass was then filtered through a Nutsche filter and the solid was washed with 6.45 liters of toluene. The wet material was taken into another reactor and 58 liters of toluene was added to it. The mass was heated to 82° C. and checked for clear dissolution. After clear dissolution was obtained, carbon treatment was given to the mass at 82° C. The mass was filtered through a candy filter in the hot condition. The carbon bed was washed with 45.15 liters of pre-filtered toluene heated to a temperature of 82° C. The combined filtrate was taken into another reactor and maintained at 28° C. for 10 hours. The mass was then filtered through a Nutsche filter and the solid was washed with 6.45 liters of toluene. The wet material was taken into a clean polythene bag. The wet material was dried in a vacuum tray drier for 14 hours under a vacuum of 690 mm Hg and a temperature of 60° C. for 14 hours to yield 9.3 kg (67.6) of the title compound.

Purity by HPLC: 99.6%

Montelukast styrene impurity: 0.03 area-%

Montelukast deschloro impurity: 0.03 area-%

Montelukast sulfoxide impurity: 0.02 area-%.

### EXAMPLE 7

### Preparation of Montelukast Sodium

92 liters of dichloromethane was taken into a reactor and 9.2 kg of montelukast tertiary butyl amine salt was added to it. The reaction mass was stirred for 10 minutes at 26° C. A solution of 1.196 kg of acetic acid in 46 liters of H ultra-filtered (“HUF”) water was prepared at 24° C. in a HDPE drum. The acetic acid solution was added to the reaction mass and stirred for 30 minutes. The organic layer was separated and the aqueous layer was extracted into 18.4 liters of dichloromethane. The combined dichloromethane layer was washed with 5×46 liters of HUF water. The dichloromethane layer was distilled under a vacuum of 500 mm Hg and a temperature varying between 18-25° C. in three hours. 18.4 liters of methanol was then added to the reactor and the reaction mass was stirred for 10 minutes. Then the methanol was distilled off to dryness under a vacuum of 600 mm Hg and at a temperature of 24° C. Another 46 liters of methanol was added to the reaction mass. In a separate reactor a solution of 0.552 kg of sodium hydroxide pellets in 46 liters of methanol was prepared. The sodium hydroxide solution was added to the above reaction mass and stirred for 20 minutes. The reaction mass was given a carbon treatment and filtered. The carbon bed was washed with 18 liters of methanol. The filtrate was subjected to ATFD at a vacuum of 720 mm Hg and a jacket temperature of 54° C. The obtained solid was dried in a vacuum tray drier at a vacuum of 670 mm Hg and a temperature of 70° C. for 14 hours to yield 6.4 kg (75.4%) of the title compound in an amorphous form.

Purity by HPLC: montelukast styrene impurity <0.006 area-%.
montelukast sulfoxide impurity <0.003 area-%.

Residual Solvent Content: Methanol 172 ppm.
Toluene 29 ppm.

### EXAMPLE 8

### Study of Hygroscopic Nature of Montelukast Sodium in Accelerated and Atmospheric Conditions

8 g portions of a montelukast sodium sample prepared according to Example 7 were kept in different environments, i.e., in accelerated and ambient conditions, and checked for water content by KF and purity by HPLC at different intervals of time. The results showed a significant increase in the water content by the Karl Fischer method in both accelerated and ambient conditions from the initial to the seventh day. There was no significant change in the HPLC purity of the compound from initial day to the seventh day.

| | | | |
|---|---|---|---|
| Duration of Study | Description | Water by KF | Purity |

Accelerated Conditions (40 ± 2° C., 75 ± 5% Relative Humidity)

| | | | |
|---|---|---|---|
| Initial day | Off-white powder | 1.5% | 99.4% |
| 1^{st} day | Pale yellow colored powder | 2.9% | — |
| 3^{rd} day | Pale yellow colored powder | 4.3% | — |
| 7^{th} day | Pale yellow colored powder | 5.4% | 99.3% |

Ambient conditions

| | | | |
|---|---|---|---|
| Initial day | Off-white powder | 1.5% | 99.4% |
| 1^{st} day | Pale yellow colored powder | 8.9% | — |
| 3^{rd} day | Pale yellow colored powder | 8.9% | — |
| 7^{th} day | Pale yellow colored powder | 8.8% | 99.4% |

### EXAMPLE 9

### Stability Study for Montelukast Sodium

Samples of montelukast sodium prepared according to Example 7 were stored and were checked for stability. Each sample was packed in a white polythene bag with nitrogen filling and tied, that bag was placed in a black polythene bag with a 1 g silica gel pouch (silica gel previously dried at 150° C. for 6 hours) with nitrogen filling and sealed, and the black bag was placed in a triple laminated bag along with a 1 g silica gel pouch (silica gel previously dried at 150° C. for 6 hours) sealed with VNS (Manufacturer: Flex Engineering Ltd. Model No. DNU-40-50-PPV-A), kept in another triple laminated bag along with a 1 g silica gel pouch (silica gel previously dried at 150° C. for 6 hours) sealed with VNS and placed in a HDPE container. The sample was analyzed for its purity and polymorphic form at monthly intervals. The results are tabulated below:

| | Moisture | Chiral Purity by HPLC | Purity by | Assay by |
|---|---|---|---|---|
| Duration | Content | (% of other isomer) | HPLC (%) | HPLC (%) |
| Initial | 1.0 | 0.02 | 99.4 | 99.5 |
| One Month | 1.2 | 0.04 | 99.4 | 99.1 |
| Two | 1.3 | 0.05 | 99.4 | 99.2 |
| Months | | | | |
| Three | 1.3 | 0.03 | 99.4 | 99.2 |
| Months | | | | |
| Six Months | 1.5 | 0.02 | 99.3 | 99.2 |

### EXAMPLE 10

### Process for the Preparation of Montelukast Styrene Impurity (Formula II)

200 ml of chloroform, 5 g of montelukast free acid and 0.8 ml of sulfuric acid were taken into a round bottom flask and the reaction mass was heated to 60° C. Water was collected azotropically from the reaction mass. The reaction mass was maintained at 60° C. for 6 hours. The reaction mass was then cooled to 28° C. 100 ml of a mixture of water and ice were charged into the reaction mass and stirred for 15 minutes. The chloroform layer was separated and washed with 50 ml of water. The chloroform layer was distilled off under a vacuum of 300 mm Hg and a temperature of 50° C. 50 ml of n-hexane was added to the crude remaining after distillation and stirred for 30 minutes. The separated solid was filtered and washed with 10 ml of n-hexane. The compound was dried at 28° C. for 8 hours to yield 4.5 g of the title compound. The structure of the compound was confirmed using NMR and Mass data.

Purity by HPLC: 94%.

1. A process for preparing amorphous montelukast sodium comprising removing solvent from a solution comprising montelukast sodium using agitated thin film drying.

2. The process of claim 1, wherein solvent is removed under reduced pressure at a temperature below the atmospheric pressure boiling point of solvent.

3. The process of claim 1, wherein solvent is removed at temperatures about 35° C. to about 60° C., under a pressure about 400 to about 740 mm Hg.

4. A process for preparing montelukast sodium, comprising:
dissolving montelukast in a solvent and recrystallizing montelukast;
reacting recrystallized montelukast with t-butyl amine to form a salt and recovering solid product;
dissolving a t-butyl amine salt of montelukast in a solvent and recrystallizing a t-butyl amine salt of montelukast; and
reacting a recrystallized t-butyl amine salt of montelukast with sodium hydroxide.

5. The process of claim 4, further comprising removing solvent from a solution comprising montelukast sodium using agitated thin film drying, to form amorphous montelukast sodium.

6. Montelukast sodium prepared by the process of either of claim 4 and containing less than about 0.5 area-% by high performance liquid chromatography of each of:
a) a compound having the formula 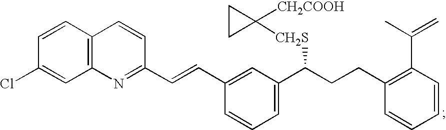
b) a compound having the formula 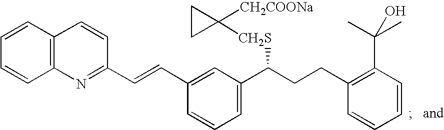
c) a compound having the formula

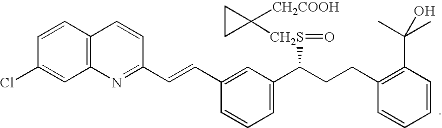

7. The montelukast of claim 6, containing less than about 0.1 area-% by high performance liquid chromatography of each of a), b), and c).

8. A method for packaging montelukast sodium, comprising:
placing montelukast sodium in a sealed container under an inert atmosphere;
placing the sealed container, a desiccant, and an oxygen adsorbent in a second sealed container;
placing the second sealed container in a triple laminated bag and sealing; and
enclosing the triple laminated bag in a closed high density polyethylene container.

9. Montelukast sodium prepared by the process of either of claim 5 and containing less than about 0.5 area-% by high performance liquid chromatography of each of:
a) a compound having the formula 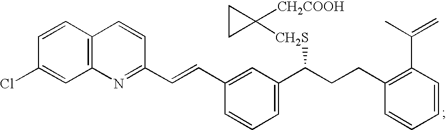
b) a compound having the formula 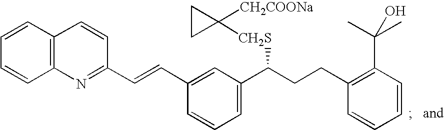
c) a compound having the formula

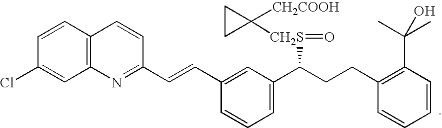

10. The montelukast of claim 9, containing less than about 0.1 area-% by high performance liquid chromatography of each of a), b), and c).
